(19) United States
(12) Patent Application Publication
(10) Pub. No.: US 2022/0387786 A1
(43) Pub. Date: Dec. 8, 2022

(54) HIGH VISUAL ACUITY, HIGH SENSITIVITY LIGHT SWITCHABLE NEURAL STIMULATOR ARRAY FOR IMPLANTABLE RETINAL PROSTHESIS

(71) Applicants: NANOVISION BIOSCIENCES, INC., La Jolla, CA (US); THE REGENTS OF THE UNIVERSITY OF CALIFORNIA, Oakland, CA (US)

(72) Inventors: Yu-Hsin Liu, San Diego, CA (US); Yu-Hwa Lo, San Diego, CA (US); Hiren D. Thacker, San Diego, CA (US); Nicholas Oesch, Del Mar, CA (US)

(21) Appl. No.: 17/833,843

(22) Filed: Jun. 6, 2022

Related U.S. Application Data
(60) Provisional application No. 63/197,239, filed on Jun. 4, 2021.

(51) Int. Cl. A61N 1/05

(52) U.S. Cl. CPC A61N 1/0543

(57) ABSTRACT

Retinal prostheses are described with visual acuity better than 20/150, and higher sensitivity, dynamic range, and FOV than the state-of-the-art. At least two different techniques are presented, the first being an optically-switched vertical single-transistor amplifier for ultrahigh photocurrent amplification, and the second being nanopatterned pillar electrodes.

This Patent application claims priority to U.S. Provisional Patent Application Ser. No. 63/197,239, filed Jun. 4, 2021; the content of which is hereby incorporated by reference herein in its entirety into this disclosure.

### BACKGROUND OF THE SUBJECT DISCLOSURE

### Field of the Subject Disclosure

The present subject disclosure relates to systems and methods for detecting light for implantable retinal prosthesis.

### Background of the Subject Disclosure

Degenerative retinal disorders are the leading cause of legal blindness (visually acuity worse than 20/200) in the United States, with age-related macular degermation (AMD) as the main cause among Hispanics and non-Hispanic Whites [1]. 11 million are affected by AMD in the US, with the current numbers projected to reach 22 million by 2050 [2]. About 8 out of 10 people with AMD have the dry form and, over time, patients become functionally blind in both eyes.

Diabetic macular edema (DME) occurs in diabetic patients when high blood sugar levels damage blood vessels which leak into the macula and can lead to permanent vision loss due to the loss of photoreceptors. Cases of DME are estimated to reach 7.8 million in 2020 in the US and Europe [4].

Retinitis pigmentosa (RP) is a rare inherited disease that is estimated to affect 100,000 people in the US [3]. While the numbers of patients affected by RP are much less than those with AMD, it is an even more devastating disorder because the typical age of diagnosis is in the late teens or early twenties. These patients are often completely and profoundly blind by their late thirties or early forties.

Finally, Stargardt disease is a rare inherited form of macular degeneration that causes progressive vision loss in children and young adults. All these forms of degenerative retinal disorders are irreversibly debilitating diseases with a substantial impact on the day-to-day quality of life for individuals as well as their families. Economically, the total amount of support required by RP and Stargardt patients over their lifetimes exceeds those of AMD patients due to age of diagnosis.

Modern implantable retinal prosthetics replace degenerated photoreceptors with optoelectronic hardware. The most common metric for quantifying the loss or restoration of vision is visual acuity (VA), with 20/20 vision accepted as normal and 20/200 as the threshold for blindness. Normal vision corresponds to an angular separation of 1 arcmin or approximately 5 μm on the retina. Correspondingly, a pixel pitch of approximately 50 μm is required for 20/200 vision.

While increasing implant pixel density is necessary to increase visual acuity, it is not sufficient. To replace the function of degenerated photoreceptors [10-15,20], these implants must be able to efficiently convert incident light into electrical current exceeding the neural stimulation threshold, and deliver this stimulating current to activate bipolar cells via the electrode/tissue interface. Applicants and other groups have shown that a stimulating current density of ˜1 mA/mm² delivered over a 1-4 ms pulse is required to stimulate a retinal bipolar cell in diseased eyes [32]. This current density is several orders of magnitude greater than the photocurrent from photodetectors illuminated by natural light [10].

Several groups (for example, Retina Implant AG and Iridium Medical Technologies) have sought to leverage CMOS (complementary metal-oxide-semiconductor) image sensor technologies in their retinal prosthesis implants [24-29]. Here, each pixel comprises a photodetector and a CMOS circuit consisting of an amplifier to produce and regulate the gain, and an output driver to produce sufficient current to stimulate bipolar neurons. However, the detector, stimulating electrode, and amplifier each occupy significant area and the latter also consumes considerable power and generates heat near sensitive ocular and retinal tissue. This limits the ability to shrink pixel size for higher visual acuity.

On the other hand, retinal prostheses from Pixium Vision and Applicants (Gen1) use cascaded photovoltaic devices and optoelectronic nanowires, respectively, without any amplification but in conjunction with a goggle accessory to produce stimulation. These goggles project pulsed high-irradiance (≥mW/mm²) images of the visual field onto the implanted sensor to produce adequate photocurrent. The projection from the goggles on to the retina through natural eye optics defines the visual field available to the patient. An implanted optoelectronic sensor typically has a smaller FOV (field of view) than the projected image, and the patient can use natural eye scanning motion to observe the visual field. This also allows for natural micro-saccadic movements used by the eye to maintain focus on objects. However, practical power density limits and optical losses in goggle/projector construction along with eye safety limits of long-term NIR exposure [33] constrain the projection FOV and limit the visual experience for the patient. To summarize, any improvements in the efficiency of converting light into stimulating current, will be immediately realized in terms of VA as well as FOV and light exposure safety margin.

### SUMMARY OF THE SUBJECT DISCLOSURE

What is presented in this subject disclosure is a retinal prosthesis with visual acuity better than 20/150, and higher sensitivity, dynamic range, and FOV than the state-of-the-art. At least two different techniques are presented, the first being an optically-switched vertical single-transistor amplifier for ultrahigh photocurrent amplification, and the second being nanopatterned pillar electrodes.

In one exemplary embodiment, the present subject disclosure is a retinal prosthesis. The prosthesis includes an array of pixels, each pixel containing a photoconductor, a vertical MOSFET amplifier, and a stimulation electrode; and a local return electrode in communication with each pixel to form a local current flow loop between the pixel, a proximal bipolar cell, and the return electrode.

In another exemplary embodiment, the present subject disclosure is a retinal prosthesis. The prosthesis includes an array of pixels, each pixel containing a partially blocked Si/Ge photoconductor, a vertical MOSFET amplifier, and a high CIC IrO stimulation electrode; and a local return electrode in communication with each pixel to form a local current flow loop between the pixel, a proximal bipolar cell, and the return electrode.

In yet another exemplary embodiment, the present subject disclosure is a retinal prosthesis. The prosthesis includes an array of pixels including pillar structure electrodes with nanopatterned stimulation surfaces; and a local return electrode in communication with each pixel to limit electric field spreading and minimize crosstalk.

### BRIEF DESCRIPTION OF THE DRAWINGS

FIG. 1A shows a subretinal implant array of compact biomimetic semiconductor optoelectronic device, with physical layout of the pixels, according to an exemplary embodiment of the subject disclosure.

FIG. 1B shows a subretinal implant array of compact biomimetic semiconductor optoelectronic device, with conceptual illustration of direct light-induced neural stimulation, according to an exemplary embodiment of the subject disclosure.

FIG. 2A shows a cross section of a pixel design, according to an exemplary embodiment of the subject disclosure.

FIG. 2B shows an equivalent circuit diagram, according to an exemplary embodiment of the subject disclosure.

FIG. 3A shows 3D electrodes, with pillar electrodes, according to an exemplary embodiment of the subject disclosure.

FIG. 3B shows 3D electrodes, with tapered pillar electrodes, according to an exemplary embodiment of the subject disclosure.

FIG. 3C shows 3D electrodes, with nanopatterned high CIC structures fabricated atop pillars, according to an exemplary embodiment of the subject disclosure.

FIG. 4A shows FET devices based on pnp epitaxial Si design, with cross-section of the pnp device structure, according to an exemplary embodiment of the subject disclosure.

FIG. 4B shows FET devices based on pnp epitaxial Si design, with drain to source current of three-electrode pnp FET devices under different gate voltage, according to an exemplary embodiment of the subject disclosure.

FIG. 5A shows an illustration of a photoconductor voltage provider, with the a-SI bar design photoconductor with three terminal contact pads, according to an exemplary embodiment of the subject disclosure.

FIG. 5B shows an illustration of a photoconductor voltage provider, with resistor model to illustrate the photoconductivity changes for L1 region, according to an exemplary embodiment of the subject disclosure.

FIG. 6A shows a material design for the voltage provider, according to an exemplary embodiment of the subject disclosure.

FIG. 6B shows another material design for the voltage provider, according to an exemplary embodiment of the subject disclosure.

### DETAILED DESCRIPTION OF THE SUBJECT DISCLOSURE

The present subject disclosure overcomes many of the drawbacks of conventional systems as described above. To develop a retinal prosthesis with visual acuity better than 20/150, and higher sensitivity, dynamic range, and FOV than the state-of-the-art, Applicants propose the following exemplary but non-limiting innovations.

(1) An optically-switched vertical single-transistor amplifier for ultrahigh photocurrent amplification: This design can yield both high sensitivity and packing density. The high sensitivity greatly reduces the required corneal irradiance level so the device could operate with standard intensity AR/VR goggles well within the long-term retinal and corneal safety limits or even under natural light (in bright sunlight), and with a large FOV. The high packing density is enabled by the unique design of the single-transistor vertical amplifier, which (a) reduces the area needed for photodetection due to high responsivity, (b) eliminates area needed for complex amplifier circuitry, and (c) shares the footprint of the stimulating electrode.

(2) Nanopatterned pillar electrodes: In recognition of the high (˜1 mA/mm²) neural stimulation threshold in diseased eyes and the CIC (charge injection capacity) limits of stimulation electrode materials (e.g., IrO), pillar electrodes are proposed here with nanopatterned stimulation surfaces. This will not only increase electrode surface area without increasing footprint, but also bring electrodes closer to the target neurons, minimizing both electrode crosstalk and stimulation threshold. The proposed pixel design also includes local (pixel-wise) return electrodes to limit electric field spreading and further minimize crosstalk.

The innovations in (1) and (2) above significantly advance the field of retinal prostheses by producing a device containing as many as 23,000 pixels at a 35 μm pitch to achieve a VA of 20/150 for a sensor FOV better than 20 degrees and a wide dynamic range. Applicants estimate this optoelectronic hardware would allow the optical power requirement from the goggles to be reduced by at least 2-orders of magnitude compared to current systems. The anticipated performance is a great leap from the state-of-the-art.

Pixel Design

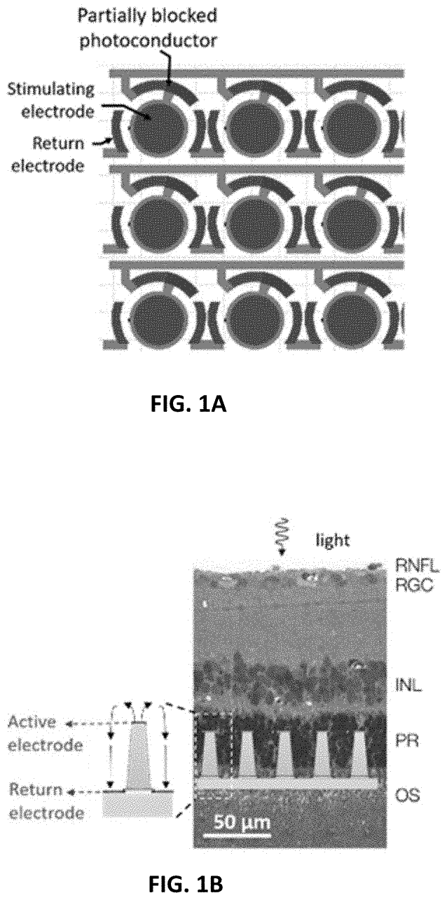

FIG. 1 presents a subretinal implant array of compact biomimetic semiconductor optoelectronic device. FIG. 1(a) Shows a physical layout of the proposed pixels each including a photoconductor, a vertical MOSFET (metal-oxide-semiconductor field-effect transistor) amplifier, and a high CIC electrode. FIG. 1(b) shows a conceptual illustration of direct light-induced neural stimulation by the optoelectronic device array in a subretinal implant. Current output from active electrodes forms an electric field towards localized return electrode. Signals from each pixel replace the original photoreceptors and are processed and relayed by the cells of the INL (Inner Nuclear Layers) to the RGCs (retinal ganglion cells). The axons of the retinal ganglion cells form the retinal nerve fiber layer (RNFL), which relays visual signals to the brain. Photoreceptors are located at the back of the eye, in contact with the retinal pigment epithelium (RPE).

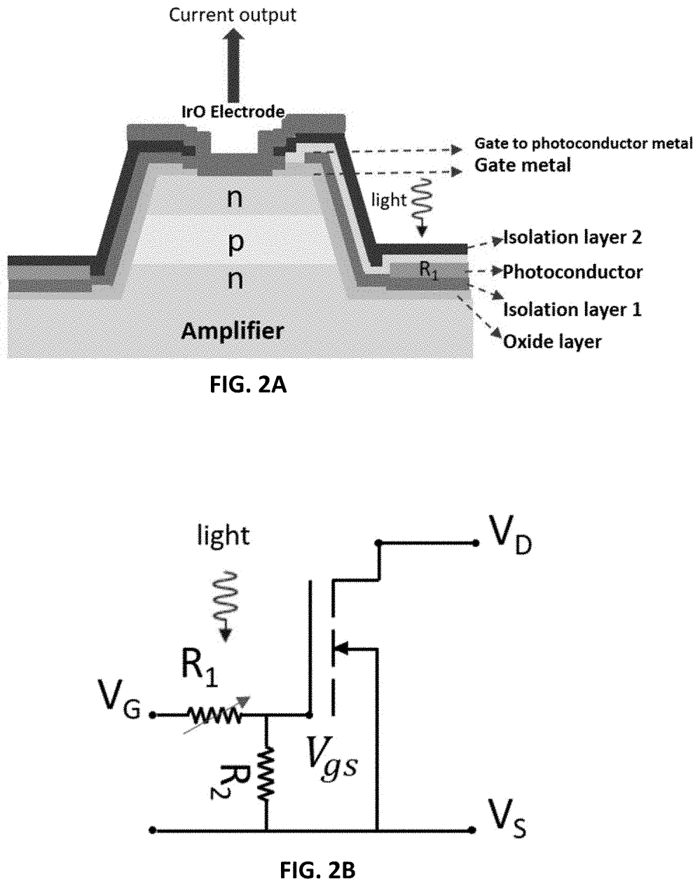

The retinal prosthesis contains a dense array of pixels each comprising a high CIC IrO stimulation electrode atop a vertical single transistor amplifier and a partially-blocked annular amorphous semiconductor photoconductor as a highly photosensitive voltage provider (see FIG. 1A). A local return electrode is placed in close proximity of each pixel to form a local current flow loop between the pixel, the proximal bipolar cell, and the return electrode, thus confining the electrical field to minimize crosstalk and increase spatial resolution (FIG. 1B). Incident light illuminates the exposed portion of the amorphous Si/Ge photoconductor, modifying the local conductivity and producing voltage division between the exposed and covered segments of the photoconductor (FIG. 1, FIG. 2). Voltage tapped from this segment drives the gate voltage to a vertical MOSFET, modulating the drain to source current with a current gain of where is the transconductance of the vertical MOSFET and is the change of the gate voltage from the output of the a-Si/Ge photoconductor. The vertical MOSFET has an effective channel length of 0.2 μm, determined by the implantation profile, and an effective gate width of 50 μm, approximately equal to the circumference of the 15 μm diameter mesa. It would produce an output current at the level of a few μA/pixel (or on the order of 10 nC for each current pulse), which is sufficient for retinal stimulation. The amount of light to switch the gate voltage via the a-Si/Ge photoconductor can be designed to be lower than 10 μW/mm², corresponding to <100 pW illumination over a photosensitive area of 10 μm². This is possible because of the low dark current in the a-Si/Ge photoconductor (in pA range). In other words, the present design can convert 100 pW light over the photosensitive area of the pixel into a current of 1-10 pA, giving rise to an effective responsivity of 10⁴-10⁵ A/W.

The output current from the vertical transistor flows through an IrO electrode that sits atop the vertical transistor area and occupies the same footprint, in a configuration that produces the most efficient use of the chip real estate. The drain current in the IrO electrode flows into the ionic buffer between the electrode and the retinal bipolar cell as Faradaic current (plus some displacement current as biphasic bias is applied to assure charge balance for each cycle of neural stimulation).

Overall, the high responsivity reduces the required light illumination level by 4 orders of magnitude compared to the cascaded photovoltaic design [12]. Importantly, the single transistor design consumes 1 uW/pixel to achieve neural stimulation, which is more power efficient than CMOS pixels [29]. These features and the efficient use of chip real estate favor high acuity, large FOV (≥20°) retinal prosthesis.

Optically-Controlled, Vertical Single-Transistor Amplifier

FIG. 2A shows a cross section of an exemplary pixel design: a vertical MOSFET as the current amplifier in the center, active electrode structure on the top, and amorphous photoconductor in the surrounding area. FIG. 2B shows an equivalent circuit diagram for the optically controlled vertical single transistor amplifier pixel with two sections of photoconductor materials to control the gate bias.

A vertical MOSFET follows the typical field-effect-transistor relation in the saturation regime as a planar device:

I_{D}=(w/2L)μnCᵢ(V_{gs}−Vₜₕ)²

where I_{D}: drain current, W: channel width (the circumference of the device mesa), L: channel length, μₙ; electron mobility (assuming n-channel FET), Cᵢ: gate capacitance. The gate voltage is controlled by an optically controlled photoconductive switch made of an amorphous Si/Ge thin film with one part of the film exposed to light and another part covered. The resistance of the exposed section and the covered section are modeled by R1 and R2 (FIG. 2B), respectively, with R1 being a function of input irradiance. The voltage at the intercept of the two sections becomes the gate voltage of the vertical transistor. We can represent V_{gs} as V_{gs}=Vₒ (R₂/(R₁(I)+R₂)), where R₁(I) represents the resistance of the exposed a-Si/Ge area. The present device is designed in such a way that R₁>>R₂ in dark condition so V_{gs}˜0 and the transistor is in the cutoff or subthreshold regime. With increased light intensity, sufficient to produce a photocurrent much greater than the dark current, R₁(I) is reduced significantly and V_{gs} Vₒ and the transistor is turned on, producing a drain current for neural stimulation. The vertical MOSFET may be configured to be an n- or p-channel FET. A SiO₂ film is formed by thermal oxidation or atomic layer deposition (ALD) on the sidewall of the silicon mesa, and the gate metal is formed by sputtering.

Next to the vertical MOSFET, an a-SiGe or a-Si thin film photoconductor, sensitive to 850 nm wavelength, is deposited on the isolation layer. An a-Si film about 1 μm thick has been previously reported that can vary its own resistance by 3 orders of magnitude from dark to 50 μW/mm² with visible light [30] owing to its high sensitivity. a-Si and a-SiGe alloys may be used to obtain the photoconductor device with the best sensitivity and controllability of the gate voltage on the vertical MOSFET. The a-Si film has strong sensitivity to green/blue light and its response drops rapidly at red and NIR wavelength. Amorphous SiGe alloys have a much stronger response at red and NIR light and would be particularly suitable for illumination from an NIR goggle. However, high Ge content in the a-SiGe alloy increases the dark current, thus reducing the sensitivity. For the present application, high responsivity to NIR light enhances photosensitivity, and a large photoconductivity change relative to the dark state gives rise to a high voltage swing, thus a high magnitude of transistor switching current. The optimal design for the Ge composition, film thickness, and photoconductor geometry for the exposed and covered sections may be deduced from experimentation.

Penetrating Pillar Electrode and Localized Return Electrode

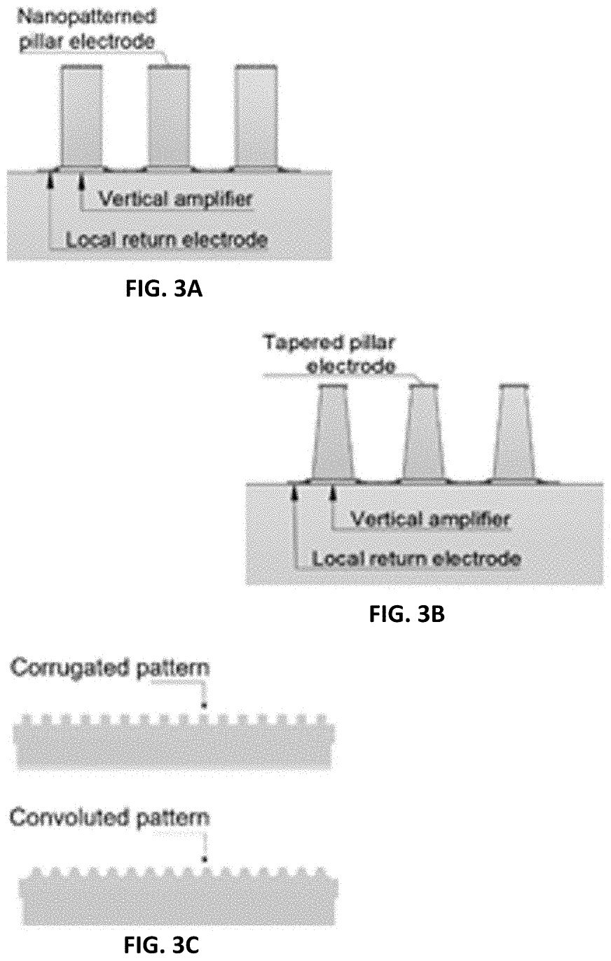

FIG. 3 shows a schematic illustration of 3D electrodes. FIG. 3A shows pillar electrodes of diameter 12-18 μm and height 30-70 μm. FIG. 3B shows tapered pillar electrodes, and FIG. 3C shows nanopatterned high CIC structures fabricated atop pillars.

In the subretinal prosthesis approach, a 30-70 μm thick layer of degenerated photoreceptors separates the implanted electrode array from bipolar cells in the INL of the retina [22,23]. According to one study [35], stimulation current threshold increases roughly proportionally to the square of the separation between the electrode and the targeted cell. Furthermore, the electric field generated by the stimulation spreads through this tissue and may stimulate multiple neurons causing pixel crosstalk and reducing VA. Thus, there are significant advantages to minimizing the distance between stimulating electrode and target neurons. Prior work [22,36] has also shown that when animal retina is placed on surfaces with topology, cells gradually migrate to fill up spaces between positive features. Even when 128 μm tall polymer structures were implanted in Yucatan minipigs, there was no significant gliosis observed or damage to the retina during implantation [22]. Therefore, an optimally designed 3D electrode, that penetrates the retina to deliver stimulation directly to the bipolar cells, and intelligently placed return electrodes to confine the electric field to individual neurons, will complement device-level advances to drive meaningful improvements to VA.

Pillar structures with diameters 12-18 μm and height ranging from 30-70 μm are fabricated on glass or silicon substrates for experimental evaluation in ex vivo animal models. The proposed 3D electrode structures are fabricated using electroplated gold and SIROF. FIG. 3 schematically illustrates variants of 3D penetrating electrodes. These include high-aspect ratio cylindrical pillars (FIG. 3A) and tapered pillars (FIG. 3B) capped with a high-CIC material. Additionally, nanopatterned corrugated and convoluted structures may be designed and fabricated on the tips of the pillars to increase effective stimulation area and charge capacity (FIG. 3C). Local return electrodes surround each pixel as shown in FIG. 1A and are connected to form a low impedance mesh return path.

Experimental Results

Bias Controlled FET Current

The center amplifier is a vertical FET, which can be configured in either a N-P-N or P-N-P configuration for a n-channel or p-channel FET. A dielectric film is deposited on the sidewall of the silicon mesa for passivation and to induce a weak inversion layer along the vertical edge of the middle layer, forming a vertical channel along the mesa sidewall. Silicon dioxide (SiO₂) or aluminum oxide (Al2O3) can be used to control the threshold voltage of the sidewall FET.

A layer of metal covers the dielectric layer as the gate terminal to control the channel. The relationship between the current output and the gate voltage can be found in a typical FET equation,

$${I}_{D} = \frac{W με s}{2 La} {( {V}_{GS} - {V}_{TH} )}^{2} ;$$

wherein the drain current I_{D} is related to the device height L and the width W of the FET (circumference of the device mesa). V_{TH} is determined by the Si epitaxial layer design and the passivation dielectric layer. I_{D} links with the V_{GS}, the gate voltage that interconnected with the surrounding photoconductor.

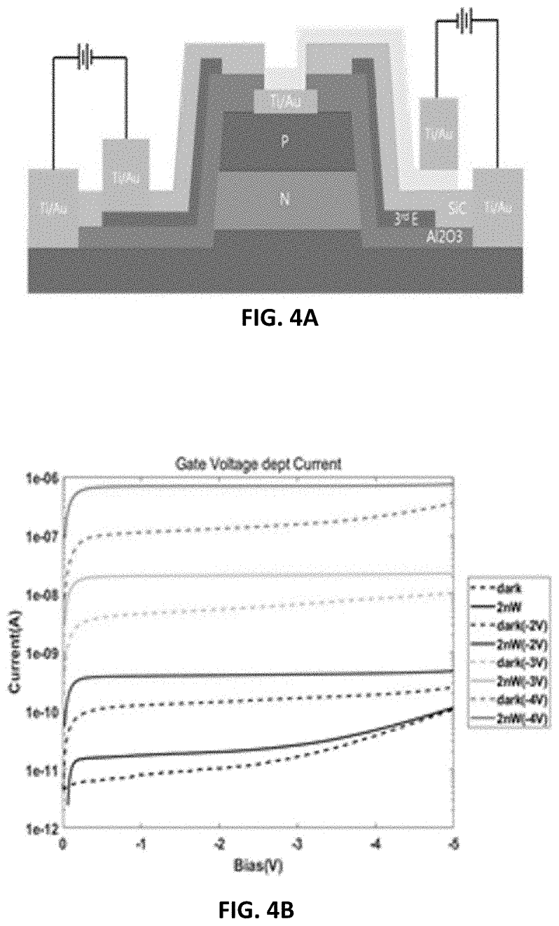

In order to adjust the amplification, the FET can also incorporate a third (gate) electrode overlying the thin passivation layer (e.g., SiO2 or Al2O3). The third electrode can be applied as a metal layer overlying the dielectric passivation shown in FIG. 4A. An additional bias can be applied on third electrode to adjust the charge of the passivation layer, thereby altering the FET threshold. The polarity of the bias applied to the third electrode can be specified depending upon the application. A negative bias across the gate-to-source of pnp structure can enhance the hole channel on the sidewall of a FET device as FIG. 4B, or a positive bias can be used to raise the channel threshold and close off the channel. On the other hand, a positive bias across gate-to-source electrodes of npn structure would enhance the electron channel on the surface while a negative bias can be used to effectively turn off the channel. The third electrode can be used to adjust the overall magnitude of output current.

FIG. 4 shows FET devices based on pnp epitaxial Si design. FIG. 4A shows cross-section of the pnp device structure with illustration of 3rd electrode on top of Al2O3 passivation layer which induces the hole channel on the sidewall. The fixed charge in the Al2O3 layer can be modulated by voltage application between 3rd electrode and the substrate. FIG. 4B shows Drain-to-Source current of three-electrode pnp FET devices under different Gate Voltage: 0V, −2V, −3V, and −4V. It also indicates that the device can be turned on by pure Gate controlling when Gate voltage is above −2V.

Optically Controlled Photoconductive Switch

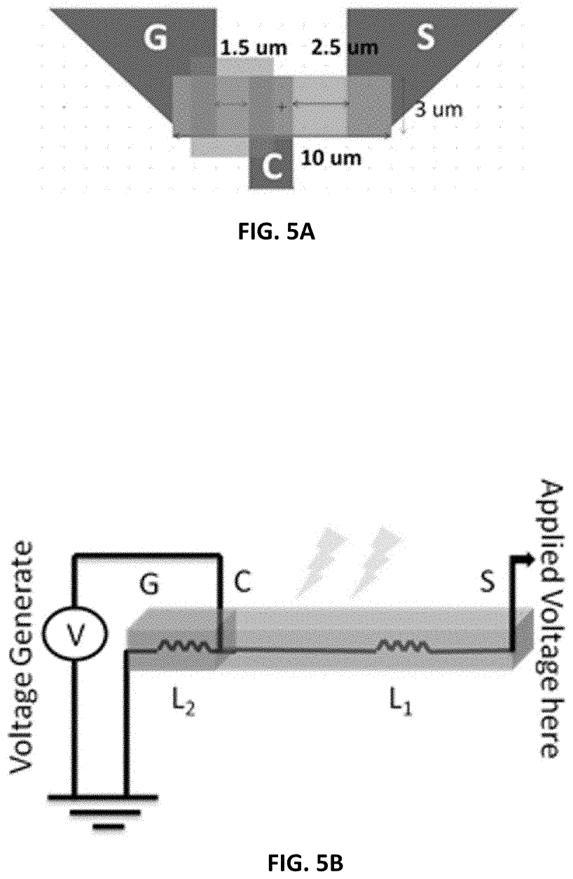

FIG. 5 shows an illustration of a photoconductor voltage provider. FIG. 5A shows the a-Si bar design photoconductor with three terminal-contact-pads G, C, and S. FIG. 5B shows a resistor model to illustrate the photoconductivity changes for L1 region. The voltage drops on L1 region changes by the intensity of light illumination and adjust the voltage output from C point.

An amorphous structure is implanted around the amplifier to provide the light sensitive voltage output to the gate of the FET. The principle of the voltage generation from an amorphous structure is demonstrated by the device shown in FIG. 5. The continuous a-Si bar has three contact pads across the structure representing Ground (G), Center (C), and Source (S) as shown in FIG. 5A. In the region between the contact pads G and C, the a-Si has been covered with a metal section to block the light illumination. In the region between contact pads C and S, a-Si is exposed to light. A photoresistor model is used in FIG. 5B to explain the light sensitive voltage output from contact pad C is obtained, the shorter L2 region represents the area covered by metal in FIG. 5A and the L1 region is the area sensitive to light. Assume a fix bias is applied across contact S and G, when there's no light (dark), since L2 length is designed to be smaller than L1 length, the voltage generated at C point should be a small value close to 0V, which is the ground. When there's light illuminated on the device, the photoconductivity of L1 increases so the voltage drop on L1 resistor is smaller than the voltage drop on L2 region, the voltage output at C point increases. By adjusting the different length ratio between L1 and L2, the best sensitive voltage output may be obtained from the contact point C to serve the gate of the main amplifier as mentioned. With the particular length design as shown in FIG. 5A, the voltage generated from C point swing from 2V to 3.5V in the dark to ambient light intensity of 0.8 uW/mm2 condition, by supplying the bias of 5V to S pad.

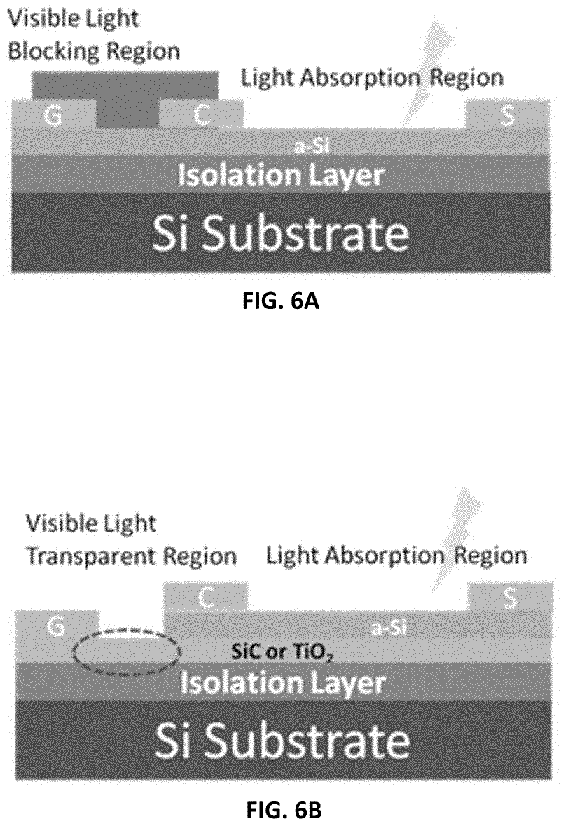

FIG. 6 shows different material design for the voltage provider. a-Si was used as the example to explain the photosensitive voltage output mechanism in the above section. Based on the applications, different amorphous materials with different absorption wavelength, such as a-Ge or a-SiGe, may be used to generate the voltage by the structure shown in FIG. 6A. For this structure, the light blocking element is required to block the light illumination between contact C and G.

Another structure as FIG. 6B to realize the light sensitive voltage provider is by using the stack of different materials. The photoconductor formed by a-Si is only at the portion between contact pad C and S, the region between contact C and G only has the material that is transparent to visible light. For example, high band gap materials such as SiC, TiO2, and ZnO are not sensitive to visible light, so there is no requirement to build in another light blocking layer on top of this area.

This application incorporates by reference herein in their entirety into this disclosure all of the following cited references, which disclose various findings as discussed in the present disclosure:
1. American Academy of Ophthalmology. https://www.aao.org/eye-disease-statistics
2. Bright Focus Foundation. https://www.brightfocus.org/
3. Foundation Fighting Blindness. https://www.fightingblindness.org/
4. J. W. Y. Yau et al., “Global prevalence and major risk factors of diabetic retinopathy,” Diabetes Care, 35, 556-564, 2012.
5. M. S. Humayun et al., “Interim results from the international trial of Second Sight's visual prosthesis,” Ophthalmology, 119, 779-788, 2012.
6. M. S. Humayun et al., “Preliminary 6 month results from the Argus II epiretinal prosthesis feasibility study,” in Conf. Proc. IEEE Eng. Med. Biol. Soc., 2009, 4566-4568.
7. J. D. Dorn et al., “The detection of motion by blind subjects with the epiretinal 60-electrode (Argus II) retinal prosthesis,” JAMA Ophthalmol., 131(2), 183-189, 2013.
8. A. K. Ahuja et al., “Blind subjects implanted with the Argus II retinal prosthesis are able to improve performance in a spatial-motor task,” Br. J. Ophthalmol., 95(4), 539-543, 2011.
9. A. C. Ho et al, “Long term results from an epiretinal prosthesis to restore sight to the blind,” Ophthalmology, 122(8), 1547-1554, 2015.
10. Y. Mandel et al., “Cortical responses elicited by photovoltaic subretinal prostheses exhibit similarities to visually evoked potentials,” Nat. Commun., 4:1980, 2013.
11. D. Boinagrov et al., “Photovoltaic pixels for neural stimulation: circuit models and performance,” IEEE Trans. Biomed. Circ. and Sys., 10(1), 2016.
12. D. Palanker et al., “Photovoltaic restoration of central vision in atrophic age-related macular degeneration,” Ophthalmology, 127(8), 1097-1204, 2020.
13. L. Wang et al., “Photovoltaic retinal prosthesis: implant fabrication and performance,” J. Neural Eng., 9(4), 2012.
14. S. Ha et al., “Towards high-resolution retinal prostheses with direct optical addressing and inductive telemetry,” J. Neural Eng., 13, 2016.
15. B. Bosse et al., “In vivo photovoltaic performance of a silicon nanowire photodiode-based retinal prosthesis,” Invest. Ophthalmol. Vis. Sci., 59(15), 5885-5892, 2018.
16. FDA HUD designation: http://www.fda.gov/ForIndustry/DevelopingProductsforRareDiseases Conditions/Designating HumanitarianUseDevices HUDS/default.htm)
17. S. Shim et al., “Retinal prosthetic approaches to enhance visual perception for blind patients,” Micromachines, 11, 535-560, 2020.
18. J. D. Weiland et al., “Retinal prosthesis,” Annu. Rev. Biomed. Eng., 7, 361-401, 2005.
19. E. Bloch et al., “Advances in retinal prosthesis systems,” Ther. Adv. Ophthalmol., 11, 1-16, 2019.
20. S. Damle et al., “Vertically integrated photo junction-field-effect transistor pixels for retinal prosthesis,” Biomed. Opt. Expr., 11, 55-67, 2020.
21. N. Oesch, “Influence of iridium oxide electrode size on stimulation thresholds and dynamic range,” presented at The Eye and The Chip, 2019.
22. J. Chen et al., “Implantation and extraction of penetrating electrode arrays in minipig retinas,” Transl. Vis. Sci. Technol., 9(5), 2020.
23. J. D. Loudin et al., “Optoelectronic retinal prosthesis: system design and performance,” J. Neural Eng., 4(1), 2007.
24. T. L. Edwards et al., “Assessment of the electronic retinal implant Alpha AMS in restoring vision to blind patients with end-stage retinitis pigmentosa,” Ophthalmology, 125(3), 432-443, 2018.
25. K. Stingl et al., “Interim results of a multicenter trial with the new electronic subretinal implant Alpha AMS in 15 patients blind from inherited retinal degenerations,” Front. Neurosci., 11, 2017.
26. K. Stingl et al., “Artificial vision with wirelessly powered subretinal electronic implant Alpha-IMS,” Proc. Biol. Soc., 280(1757), 2013.
27. R. Daschner et al., “Laboratory and clinical reliability of conformally coated subretinal implants”, Biomed. Microdevices, 19(7), 2017.
28. F. Yang et al., “Flexible, high-density microphotodiode array with integrated sputtered iridium oxide electrodes for retinal stimulation,” Journ. Micro/Nanolith., MEMS and MOEMS, 15(1), 2016.
29. C. L. Lee et al., “A 0.8-V 4096-pixel CMOS sense-and-stimulus imager for retinal prosthesis,” IEEE Trans. Electron Devices, 60, 1162-1168, 2013.
30. F. Dong et al., “Thin film amorphous silicon nanoscale photodetectors,” Procedia Chemistry, 1, 433-436, 2009.
31. X. Zhang et al., “Characterization of a light switchable microelectrode array for retinal prosthesis,” Appl. Phys. Lett., 99, 253702, 2011.
32. A. Coma et al., “Electrode-size dependent thresholds in subretinal neuroprosthetic stimulation,” J. Neural Eng., 15, 2018.
33. H. Lorach et al., “Retinal safety of near infrared radiation in photovoltaic restoration of sight,” Biomed. Opt. Expr., 7(1), 2016.
34. S. F. Cogan, “Neural stimulation and recording electrodes,” Annu. Rev. Biomed. Eng., 10, 275-309, 2008.
35. D. Palanker et al., “Design of a high-resolution optoelectronic retinal prosthesis,” J. Neural Eng., 2, 105-120, 2005.
36. E. Ho et al., “Characteristics of prosthetic vision in rats with subretinal flat and pillar electrode arrays,” J. Neural Eng., 16, 2019.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. For example, the example measurements and values presented in the disclosure are not limiting of the subject matter, but merely show an example that has been used. It would be apparent to one having ordinary skill in the art that some variation and range is possible and expected with each of the variables presented, and which would result in the desired outcome. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A retinal prosthesis, comprising:
an array of pixels, each pixel containing a photoconductor, a vertical MOSFET amplifier, and a stimulation electrode; and
a local return electrode in communication with each pixel to form a local current flow loop between the pixel, a proximal bipolar cell, and the return electrode.

2. The retinal prosthesis in claim 1, wherein the stimulation electrode has a high CIC.

3. The retinal prosthesis in claim 1, wherein the stimulation electrode comprises IrO.

4. The retinal prosthesis in claim 1, wherein the photoconductor is partially blocked.

5. The retinal prosthesis in claim 4, wherein the photoconductor is annular.

6. The retinal prosthesis in claim 5, wherein the photoconductor is amorphous.

7. The retinal prosthesis in claim 6, wherein the photoconductor comprises Si/Ge.

8. The retinal prosthesis in claim 1, wherein the MOSFET amplifier has an effective channel length of 0.2 um.

9. The retinal prosthesis in claim 8, wherein the MOSFET amplifier has an effective channel width of 50 um.

10. The retinal prosthesis in claim 1, wherein the retinal prosthesis is adapted such that 100 pW of light over the pixel is converted into a current of 1-10 μA, giving rise to an effective responsivity of 10⁴-10⁵ A/W.

11. A retinal prosthesis, comprising:
an array of pixels, each pixel containing a partially blocked Si/Ge photoconductor, a vertical MOSFET amplifier, and a high CIC IrO stimulation electrode; and
a local return electrode in communication with each pixel to form a local current flow loop between the pixel, a proximal bipolar cell, and the return electrode.

12. The retinal prosthesis in claim 11, wherein the photoconductor is annular.

13. The retinal prosthesis in claim 12, wherein the photoconductor is amorphous.

14. The retinal prosthesis in claim 11, wherein the MOSFET amplifier has an effective channel length of 0.2 um.

15. The retinal prosthesis in claim 14, wherein the MOSFET amplifier has an effective channel width of 50 um.

16. A retinal prosthesis, comprising:
an array of pixels including pillar structure electrodes with nanopatterned stimulation surfaces; and
a local return electrode in communication with each pixel to limit electric field spreading and minimize crosstalk.

17. The retinal prosthesis in claim 16, wherein the pillar structure has a diameter of 12-18 um.

18. The retinal prosthesis in claim 17, wherein the pillar structure has a height of 30-70 um.

19. The retinal prosthesis in claim 16, wherein the pillar structure is cylindrical or tapered.

20. The retinal prosthesis in claim 16, wherein the pillar structure has a tip with a nanopatterned corrugated pattern or convoluted pattern.
